(19) United States
(12) Patent Application Publication
(10) Pub. No.: US 2021/0170151 A1
(43) Pub. Date: Jun. 10, 2021

(54) Microneedle Array Device, Methods Of Manufacture And Use Thereof

(71) Applicant: NanoCav, LLC, Culver City, CA (US)

(72) Inventor: Chihwei CHANG, Culver City, CA (US)

(21) Appl. No.: 16/761,359

(22) PCT Filed: Nov. 6, 2018
(86) PCT No.: PCT/US2018/059314

§ 371 Date: May 4, 2020

Related U.S. Application Data
(60) Provisional application No. 62/582,824, filed on Nov. 7, 2017.

(51) Int. Cl. A61M 37/00; A61L 31/10; A61L 31/04; A61L 31/16; B33Y 10/00; B33Y 80/00; B29C 41/04; B29C 41/22; B29C 33/56

(52) U.S. Cl. CPC A61M 37/0015; A61M 37/0076; A61L 31/10; A61L 31/041; A61L 31/16; B33Y 10/00; B29K 2067/04; B29C 41/04; B29C 41/22; B29C 33/56; A61M 2037/0023; A61M 2037/0053; A61M 2037/0061; B33Y 80/00

(57) ABSTRACT

Systems and methods for using microneedle arrays to deliver bioactive compounds are presented. In general, the microneedle array comprises at least three layers: a base layer, a separation layer, and a bioactive layer, wherein the separation layer is situated between the base layer and the bioactive layer. Upon exposure to physiological conditions, the separation layer dissolves and/or disperses, allowing the base layer to be removed while the bioactive layer remains embedded in the outer surface of the skin.

### FIELD OF THE INVENTION

Delivery platforms for transdermal delivery of bioactive compounds are provided. More specifically, microneedle arrays for delivering bioactive compounds to a patient are provided.

### BACKGROUND

The background description includes information that may be useful in understanding the subject matter disclosed herein. It is not an admission that any of the information provided herein is prior art or relevant to the subject matter disclosed herein, or that any publication specifically or implicitly referenced is prior art.

Where a definition or use of a term in an incorporated reference is inconsistent or contrary to the definition of that term provided herein, the definition of that term provided herein applies and the definition of that term in the reference does not apply.

Hypodermic needles or transdermal patches are frequently used for transdermal administration of pharmaceuticals and other compounds. However, both of these approaches have limitations. Hypodermic needles are associated with pain at the injection site and carry a risk of infection from bloodborne pathogens. Some studies estimate that up to 40% of world-wide hypodermal injections occur with reused needles, placing millions of people at risk of acquiring diseases such as hepatitis B, hepatitis C and/or HIV infections (Gill et al., Clin J. Pain (2008) 24(7): 585-594). While transdermal delivery using patches has fewer risks, this approach is also limited due to the formidable transport barrier of the stratum corneum, a physical barrier of the skin. The stratum corneum restricts the types of compounds that are able to be effectively delivered using transdermal patches to lipophilic molecules of relatively small size with relatively low dose requirements, rendering many types of compounds incompatible with transdermal delivery.

Various types of microneedle array devices have been developed as an alternative to injection or transdermal delivery. For example, some microneedle array devices are surface-coated with a bioactive compound. However, because this approach relies on surface area, the amount of bioactive compound that can be administered is limited. Other approaches use hollow microneedles attached to a reservoir of bioactive compound(s) for delivery. However, these systems are often complex to use and susceptible to mechanical malfunction, user error and contamination. Still other approaches use microneedle arrays that are dissolvable. For example, U.S. Pat. Appln. 2015/0126923 fabricates microneedles using a single type of polymer, but this approach suffers from complexities and uncertainty in administration and handling.

Thus, even though various systems and methods of administering compounds using microneedle arrays have been developed, there is still a need to provide improved systems and methods to quickly and efficiently deliver compounds transdermally.

### SUMMARY OF THE INVENTION

The present techniques are directed to various systems and methods of transdermal delivery of bioactive compounds, and in particular, to utilizing microneedle arrays for delivering bioactive compounds to a patient.

In an aspect, a microneedle array device for transdermal injection is contemplated comprising a plurality of microneedles, wherein each microneedle comprises: a base layer; a bioactive layer comprising one or more bioactive compounds; and a separation layer comprising one or more compounds that dissolve or disperse under physiological conditions, wherein the separation layer is situated between the base layer and the bioactive layer. In another aspect, a method of fabricating the microneedle array device comprising the one or more bioactive compounds for transdermal injection is provided. In yet another aspect, a method of administering the one or more bioactive compounds is provided. In still another aspect, the microneedle array device may comprise one or more bioactive compounds that induces chemotaxis in lymphocytes (e.g., NK cells, T cells) or other cells of the immune system.

Microneedle arrays as disclosed herein have a variety of advantages over transdermal patches, syringes, and other approaches. By delivering the bioactive compound(s) using a bioactive layer, the amount of material deposited into the skin can be greatly reduced. Rather than implanting the entire microneedle into the skin, the present techniques allow for the base layer to be removed, leaving behind the bioactive layer. The bioactive compound(s) may be released as the polymer associated with the bioactive layer degrades. The microneedle arrays are single administration devices and therefore reduce the risk of transmitting infections. The arrays also are likely to reduce pain at the site of injection.

Various objects, features, aspects and advantages of the inventive subject matter will become more apparent from the following detailed description of preferred embodiments.

### BRIEF DESCRIPTION OF THE DRAWINGS

FIGS. 1A-1D are illustrations showing examples of microneedles and microneedle arrays, according to the devices and techniques presented herein.

FIGS. 2A-2B are illustrations showing an exemplary mode of operation of a microneedle of a microneedle array, according to the devices and techniques presented herein.

FIG. 3 is an illustration showing a chemokine gradient in a microneedle array, according to the devices and techniques presented herein.

FIGS. 4A-4B are illustrations and images showing various aspects of an exemplary fabrication process for making the microneedles arrays, according to the devices and techniques presented herein.

FIGS. 5A-5F are illustrations and experimental images showing dissolution of the separation layer, according to the devices and techniques presented herein.

FIGS. 6A-6D are experimental images of the base layer, the separation layer, and the bioactive layer, according to the devices and techniques presented herein.

FIGS. 7A-7D are illustrations and experimental images showing microarrays implanted in agarose, according to the devices and techniques presented herein.

FIG. 8 is a series of experimental images showing degradation of the bioactive layer, according to the devices and techniques presented herein.

The examples presented herein are not intended to be limiting. It is understood that many different variations of these examples are disclosed within the application, and that all such embodiments fall within the scope of devices and methods disclosed herein.

### DETAILED DESCRIPTION

Various systems and methods of utilizing microneedle arrays are provided for delivering bioactive compound(s) to a patient. The microneedle comprises at least three layers, including a base layer, a separation layer, and a bioactive layer, wherein the separation layer is situated between the base layer and the bioactive layer. The microneedle array is embedded into a surface (e.g., the skin of a patient), wherein the separation layer dissolves under physiological conditions, allowing the bioactive layer to remain embedded in the skin of the patient, while the base layer is removed.

Dissolvable microneedle arrays provide a mechanism to provide a controlled amount of bioactive compound(s) to a specific delivery site in an efficient manner. In general, the bioactive layer, the separation layer and a portion of the base layer are embedded into a surface (e.g., the skin) of the patient. The separation layer dissolves under physiological conditions, leaving the bioactive layer embedded in the skin, and allowing the base layer to be removed. Unless indicated otherwise, physiological conditions refers to exposure to a temperature between about 20° C. to 45° C., between about 34-40° C., or between about 36-37° C., and/or exposure to an aqueous based solution.

The microneedle arrays disclosed herein have sufficient mechanical strength to puncture or penetrate the outer layer of skin, the stratum corneum. The force needed to penetrate the skin is determined by the radius and angle of the microneedle tip, in this case, determined by the geometry of the bioactive layer. The microneedle array is fabricated from materials sufficient to, and under conditions suitable for, creating arrays that are capable of puncturing human skin.

By concentrating the bioactive component(s) in the bioactive layer of the microneedle array, the bioactive component(s) can be delivered in a targeted, efficient manner, and the base layer, devoid of bioactive compound(s) can be removed. In some aspects wherein the microneedle array is used to treat skin disorders or cancers, the shape of the skin disorder or cancer can be mapped to the microneedle array, in order to precisely deliver the bioactive compounds to the site of the disorder or cancer, while not delivering bioactive compounds to nearby healthy tissue. Such an approach also minimizes deposition of non-bioactive components (e.g., the polymers used to fabricate the microneedle array) into the recipient. For recipients receiving ongoing treatments, each treatment delivered via a microneedle array additively introduces non-bioactive components into the skin. Thus, minimizing the total amount of non-bioactive components delivered to a patient provides an advantage over other types of microneedle array devices used to deliver biologics or pharmaceuticals to a recipient.

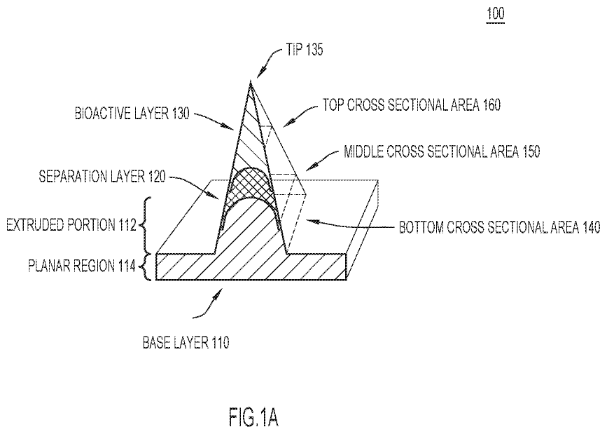

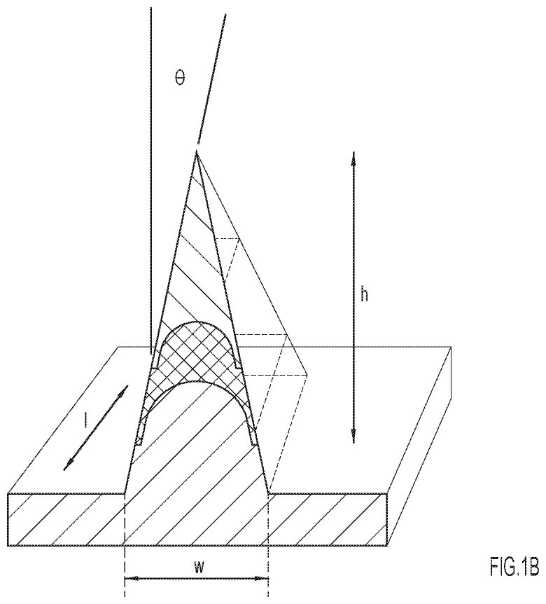

FIGS. 1A-1D show various examples of microneedles and microneedle arrays. FIG. 1A shows an example shape of a single microneedle. In this example, the microneedle 100 comprises three layers: base layer 110, separation layer 120, and bioactive layer 130 comprising a tip 135 of the microneedle.

Base layer refers to layer 110, which forms the foundation of the microneedle array. In some embodiments, the base layer may be in direct contact with the separation layer 120. In other embodiments, one or more other layers may be interposed between the base layer 110 and the separation layer 120. The base layer 110 forms a substantially planar sheet or region 114, having a corresponding thickness, and connecting the array of microneedles. The base layer may extrude into the pyramidal microneedle itself, shown as extruded portion 112 in FIG. 1A.

In this example, microneedle 100 has the overall shape of a pyramid, with each layer (starting from the base layer and ending at the bioactive layer) having an associated decrease in the cross sectional area. In this embodiment, the bottom cross sectional area 140 of the base layer is greater than the middle cross sectional area 150 of the separation layer, and the middle cross sectional area 150 is greater than the top cross sectional area 160 of the bioactive layer. It is understood that each layer, due to the pyramidal shape of the device, has a range of cross-sectional areas, and for the purposes of this discussion, it is understood that the cross-sectional area for a layer refers to the cross-sectional area at a midpoint of the layer. In this example, the base layer 110 has a portion that is substantially planar, planar region 114, and a portion that extrudes into the pyramidal shape (extruded portion 112) to form part of the microneedle. The cross-sectional area of the base layer is with reference to the extruded portion 112.

In general, the base layer 110 may be formed from one or more polymers that are not readily dissolvable under physiological conditions, e.g., polydimethylsiloxane (PDMS). It is understood that a variety of other materials may be used in lieu of PDMS to form the base layer. In some embodiments, the material for the base layer is selected based on suitability with the corresponding microfabrication process used to form the microneedle array.

A variety of materials are suitable for use as base layer 110, including but not limited to: acrylic, styrene-methyl methacrylate copolymers, ethylene/acrylic acid, acrylonitrile-butadiene-styrene (ABS), AB S/polycarbonate, ABS/polysulfone, ABS/polyvinyl chloride, ethylene propylene, ethylene vinyl acetate (EVA), nylons (including nylon 6, nylon 6/6, nylon 6/6-6, nylon 6/9, nylon 6/10, nylon 6/12, nylon 11 and nylon 12), polyacrylate, polybutylene terephthalate (PBT), polycarbonate, polyethylene terephthalate (PET), polyethylene (including low density, linear low density, high density, cross-linked and ultra-high molecular weight grades), polypropylene homopolymer, polypropylene copolymers, polyolefins, polystyrene (including general purpose and high impact grades), polytetrafluoroethylene (PTFE), ethylene-tetrafluoroethylene (ETFE), polyvinyl fluoride (PVF), polyvinylidene fluoride (PVDF), polychlorotrifluoroethylene (PCTFE), polyethylene-chlorotrifluoroethylene (ECTFE), polymethyl methacrylate (PMMA), silicon styrene-acrylonitrile (SAN), elastomers, metal oxides, plastics, and glass, or any combination thereof. Materials capable of being fabricated with sub-micron resolution, e.g., having features below 0.1 μm, such as PDMS, and that do not readily degrade upon exposure to physiological conditions are suitable for use as the base layer. Many such materials are known within the art, and all such materials are contemplated for use herein.

The separation layer is a middle layer of the microfluidics device, situated between the bioactive layer and the base layer. The separation layer dissolves upon contact with physiological conditions. Suitable materials for the separation layer preferably include polyvinyl alcohol (PVA) and/or polyvinyl pyrrolidone (PVP), but may also include other dissolvable materials known in the art, including adhesives, glues. In general, it is understood that any dissolvable material may be used to form the separation layer, provided that the dissolution occurs on a suitable timescale, e.g., within minutes. Dissolution of the separation layer occurs more quickly than biodegradation of the bioactive layer, allowing the bioactive layer to remain embedded while the base layer is removed. Many materials suitable for forming the separation layer are known within the art, and all such materials are contemplated for use herein.

Bioactive layer refers to a layer of the microneedle containing one or more bioactive compounds. Bioactive compound refers to a molecule having an effect on a living organism, e.g., one or more biological activities. Bioactive compounds are administered to a patient using the microneedle array. Biological activities include but are not limited to, inhibiting or activating a biological process, regulating a biological process, catalyzing an enzymatic reaction, triggering an immune response, inducing chemotaxis, inhibiting or activating cellular proliferation, etc.

In some embodiments, bioactive compounds include but are not limited to antibodies or fragments thereof, affimers, allergens, analgesic agents, anesthetic agents, anti-asthmatic agents, antibiotics, anti-depressant agents, anti-diabetic agents, anti-fungal agents, antigens (with or without adjuvants), anti-hypertensive agents, anti-inflammatory agents, anti-neoplastic agents, aptamers, bacteria, chemotaxis agents, chemotherapeutic agents, cosmetics (e.g., anti-aging compounds, anti-wrinkle compounds, dermal fillers, ink for tattoos, glow in the dark compounds, UV absorbent compounds, skin brightening compounds, etc.), diagnostic agents, DNA, glycoproteins, immunostimulating agents, immunosuppressive agents, lipids, nucleic acid constructs, nucleotides, oligonucleotides, oligosaccharides, peptides, polysaccharides, proteins, protein scaffolds, RNA, small molecules, vaccines, vaccines with adjuvants, vectors, viral vectors, viruses (live or inactivated), wound healing drugs/agents, birth control drugs, and nanoparticles, etc. It is specifically contemplated that the microneedle may be formulated using any suitable biologic or pharmaceutical for delivery to a subject.

The bioactive layer typically forms the tip of the microneedle. When the microneedle array is brought into contact with the skin, the tip of the microneedle pierces the skin. The bioactive layer separates from the microneedle array base layer, where it remains embedded into the dermal layer after the base layer is removed.

Accordingly, the bioactive layer comprises a material that is biodegradable under physiological conditions, a material that is insoluble but dispersible under physiological conditions, or a material that includes a combination thereof. For example, the bioactive layer may be formed using any suitable material (e.g., polymers such as poly(lactic-co-glycolic acid (PLGA), carboxymethylcellulose (CMC), etc., or a mixture thereof). The bioactive layer may further comprise micro- or nano-particles comprising absorbed, conjugated, dispersed, or encapsulated bioactive compounds, wherein the micro- or nano-particles are designed to deliver the bioactive compound to the subject. Many materials suitable for forming the bioactive layer are known within the art, and all such materials are contemplated for use herein.

When the microneedle is embedded into the skin of a subject, the bioactive layer biodegrades, resulting in delivery of the bioactive compound(s) into the underlying dermal tissue of the subject. While both the separation layer and bioactive layer are dissolvable/dispersible or biodegradable, respectfully, it is understood that the rate of dispersal or dissolution for the separation layer occurs more rapidly than biodegradation for the bioactive layer, allowing the base layer to be removed and the bioactive layer to remain embedded in the dermal layer of the patient. The bioactive layer may be formulated to release the embedded bioactive compound(s) over a desired period of time (e.g., hours, days, etc.).

In the examples provided herein, PLGA is utilized in the formation of the bioactive layer. In some embodiments, PLGA may require relatively high temperatures (e.g., greater than 135° C.) for fabrication, while the bioactive compounds may include compounds that are not stable at high temperatures (e.g., nucleic acids, peptides, proteins, vaccines, viruses, etc.). Accordingly, other materials, which may be formed at lower temperatures (e.g., CMC) and are biodegradable may be used in lieu of PLGA.

The bioactive layer can be formulated to include multiple bioactive compounds in a single microneedle or in a portion of the microneedle array. Thus, it is specifically contemplated that the microneedle array can deliver two or more bioactive compounds to a patient. In some embodiments, the microneedle array can deliver immuno-chemotherapeutic treatments to a patient by co-delivering bioactive compounds, e.g., a cytotoxic agent such as a chemotherapeutic with an immune modulatory compound, e.g., a checkpoint inhibitor; or a checkpoint inhibitor with a chemotaxis agent, to alleviate immune system evasion while promoting migration of NK cells to the site of the tumor.

Referring to FIG. 1B, the microneedle, and in particular, the pyramidal extrusion of the microneedle, has an associated length (l), width (w), and height (h) along with an angle (Θ) reflecting the sharpness of the needle. The length and the width may also be referred to as in plane dimensions and the height may be referred to as an out of plane dimension.

In some embodiments, the width of the microneedle ranges from about 10 μm to about 1000 μm or more. For example, the width of the microneedle may be about 10 μm, 15 μm, 20 μm, 25 μm, 30 μm, 35 μm, 40 μm, 45 μm, 50 μm, 55 μm, 60 μm, 65 μm, 70 μm, 75 μm, 85 μm, 90 μm, 95 μm, 100 μm, 200 μm, 300 μm, 400 μm, 500 μm, 600 μm, 700 μm, 800 μm, 900 μm, 1000 μm, and so forth, or any value in between.

In other embodiments, the length of the microneedle may range from about 10 μm to about 1000 μm. For example, the length of the microneedle may be about 10 μm, 15 μm, 20 μm, 25 μm, 30 μm, 35 μm, 40 μm, 45 μm, 50 μm, 55 μm, 60 μm, 65 μm, 70 μm, 75 μm, 85 μm, 90 μm, 95 μm, 100 μm, 200 μm, 300 μm, 400 μm, 500 μm, 600 μm, 700 μm, 800 μm, 900 μm, 1000 μm, and so forth, or any value in between.

In still other embodiments, the height of the microneedle may range from about 50 μm to about 2500 μm. For example, the height of the microneedle may be about 50 μm, 75 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 800 μm, 900 μm, 1000 μm, 1100 μm, 1200 μm, 1300 μm, 1400 μm, 1500 μm, 1600 μm, 1700 μm, 1800 μm, 1900 μm, 2000 μm, 2100 μm, 2200 μm, 2300 μm, 2400 μm, 2500 μm, and so forth, or any value in between.

The angle of the tip (Θ) may range from about 10° to 85° degrees. For example, the angle may be about 10°, 15°, 20°, 25°, 30°, 35°, 40°, 45°, 50°, 55°, 60°, 65°, 70°, 75°, 80° or 85°, or any value in between.

In this example, the needle has a pyramidal shape. In other examples, other shapes may be used. The length of the microneedle may be greater than the width of the microneedle. For example, the length of the needle may be about 1.1 to 5, about 1.1 to 4, about 2 to 4, about 2 to 3, or about 3 to 4 times greater than the width of the microneedle. In other embodiments, the width of the microneedle is the same as or about the same as the length of the microneedle. In still other embodiments, the width of the microneedle may be greater than the length of the microneedle. For example, the width of the needle may be about 1.1 to 5 times, about 1.1 to 4 times, about 2 to 4 times, about 3 to 4 times, or about 2 to 3 times greater than the length of the microneedle.

FIG. 1C shows a microneedle array of six microneedles 100(1)-100(6). Microneedle arrays can be of any suitable size, having any number of rows and any number of columns. In general, multiple rows, each row having a plurality of microneedles, are present in the microneedle array. In some embodiments, the number of microneedles in a microneedle array ranges from 2 to about 1000, or more. In some embodiments, the number of microneedles in a microneedle array ranges from about 10 to about 200, or from about 40 to about 100, or more.

In some embodiments, within a row of microneedles, the individual microneedles may be spaced at an interval (dx) apart from each other, wherein the interval is regularly spaced or fixed. In this example, the interval (dx) refers to a distance from the extruded portion of the base of a first microneedle, e.g., 100(1), to the extruded portion of the base of a second microneedle, e.g., 100(2). For example, a microneedle may be placed every 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 85 μm, 90 μm, 95 μm, 100 μm, and so forth, including any value in between.

In some embodiments, the posts are arranged at an interval (dy), wherein the interval is regularly spaced or fixed. In this example, the interval (dy) refers to a distance from the extruded portion of the base of a first microneedle in a first row to the extruding portion of the base of a second microneedle in a second row, assuming an offset (xo) of zero. For example, a microneedle may be placed every 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 85 μm, 90 μm, 95 μm, 100 μm, and so forth, including any value in between.

In other embodiments, successive rows of posts may have an offset (xo). For example, a row (r) may be established at a particular location (e.g., using the Cartesian coordinate system x, y). The (r+1)th row may be displaced by an amount (xo). The (r+2)th row (not shown) may be displaced to the right by an amount (2xo), and so forth. Offsets may occur in a fixed manner, such that each successive row is displaced in the same direction by a fixed amount. Alternatively, offsets may occur in a variable manner, such that each successive row is displaced in the same direction by a variable amount. In other embodiments, an offset may be applied in an alternating direction, such that with regard to a row (r), the (r+1)th row has a fixed or variable offset in one direction, and the (r+2)th row has a fixed or variable offset in the opposite direction (e.g., one offset is to the right and the next offset is to the left).

In some embodiments, the total length (tl) and the total width (tw) of the microneedle array ranges from about 1 to 100 mm by about 1 to 100 mm. In some embodiments, the 2D arrangement of the microneedle array is square, rectangular or circular, but may also be semi-circular, V-shaped, or any other appropriate shape suitable for administration to a surface of the patient. In still other embodiments, each microneedle array includes about 10 to 1000 microneedles or more.

Referring to FIG. 1D, the microneedles may be formed in any shape suitable for insertion into the dermal layer. In this example, the base layer 112, the separation layer 122 are both cylindrical and the bioactive layer 132 is conical. In this embodiment, the bottom cross sectional area 142 is the same or is about the same as the middle cross sectional area 152, and the middle cross sectional area 152 is greater than the top cross sectional area 162. In this example, the diameter of the microneedle (twice the radius) ranges from about 10 μm to 1000 μm, while the height of the microneedle ranges from about 50 μm to 2500 μm. The angle of the tip may range from 10° to 85°.

The microneedle can take on a variety of shapes. In general, the microneedle may be formed based on any shape (as viewed from the top-down orientation, and with respect to dimensions (w) and (l), including but not limited to, circular, oval, square, diamond, rectangular, etc. provided that the tip of the microneedle has a suitable geometry in which to puncture the skin.

In other embodiments, the microneedle has a pyramidal shape. In other embodiments, the microneedle has a base layer and a separation layer that is conical, with a bioactive layer that is pyramidal, wherein sides of the pyramid are flat or curved/arcuate to facilitate insertion into the skin. Here it is understood that many different type of geometries are suitable and all are within the scope of the embodiments described herein.

In some embodiments, the dimensions of microneedle arrays and the positioning of the microneedles depends upon the application. For example, the shape of a region (e.g., a cancerous growth or lesion) on a patient's skin may be mapped to a microneedle array to ensure that the shape of the microneedle array is sufficient to cover the entire region. This helps ensure that the entire region receives the appropriate amount of bioactive compound. In other examples, the microneedle array may contain cosmetics (e.g., anti-aging compounds, anti-wrinkle compounds, dermal fillers, ink for tattoos, skin brightening compounds, etc.) for application to a specific region of the skin (e.g., under the eyes, forehead, nose, etc.). Similarly, the shape of the target region can be mapped to the microneedle array to ensure that the entire region of the face receives suitable treatment. In still other embodiments, the techniques disclosed herein can be used to deliver anti-bacterial compositions to skin infections that are difficult to treat with traditional oral antibiotic therapy, such as MRSA. By delivering the antibiotic(s) directly to the site of infection, in a form that is released over a suitable timeframe, the likelihood of successful treatment is increased. In still other embodiments, diseases which require prolonged administration of a medication, e.g., tuberculosis, may benefit from the techniques presented herein. Instead of administering a daily dose of an antibiotic, the antibiotic may be implanted underneath the skin. This reduces the likelihood of antibiotic resistance, as the antibiotic is administered at a relatively constant dose (as a function of biodegradation), and the possibility of missing doses is greatly reduced. Accordingly, the microneedle array can be topologically configured to deliver specific ingredients to specific locations on a patient's skin to achieve a desired cosmetic benefit or therapeutic effect.

In other embodiments, the bioactive layer comprises one or more additional ingredients, e.g., to stabilize or preserve the activity of the bioactive compound, to modulate the rate of release of the bioactive compound, to maintain sterility of the biologic or pharmaceutical, etc. These ingredients include but are not limited to: antioxidants, bacteriostats, buffers, carbohydrates, chelating agents such as EDTA or glutathione, coloring, diluents, emulsifiers, excipients, flavoring and/or aromatic substances, lubricants, pH buffering agents, physiologically acceptable carriers, polypeptides (e.g., glycine), preservatives, proteins, salts for influencing osmotic pressure, solubilizers, stabilizers, surfactants, wetting agents, etc. Buffers include but are not limited to saline, neutral buffered saline, phosphate buffered saline, etc. Carbohydrates include but are not limited to dextrans, glucose, mannose, mannitol, sucrose, etc. Stabilizing excipients include but are not limited to sugars, carbohydrates and various polymers.

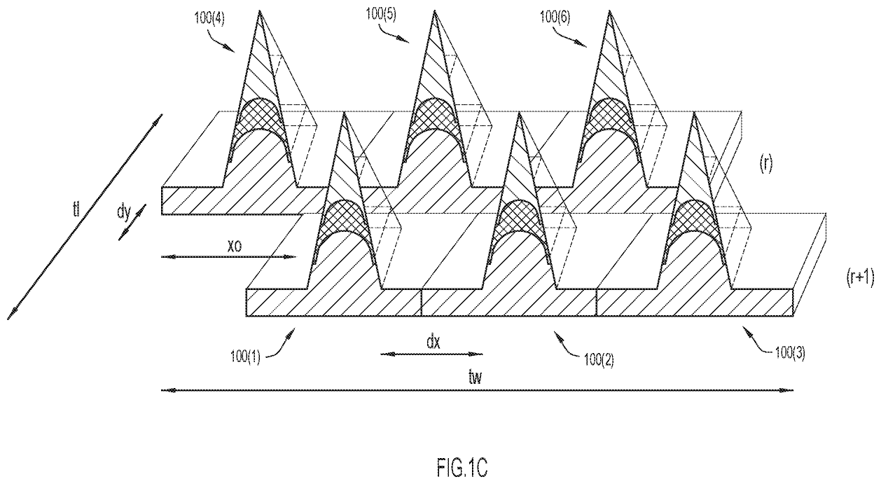

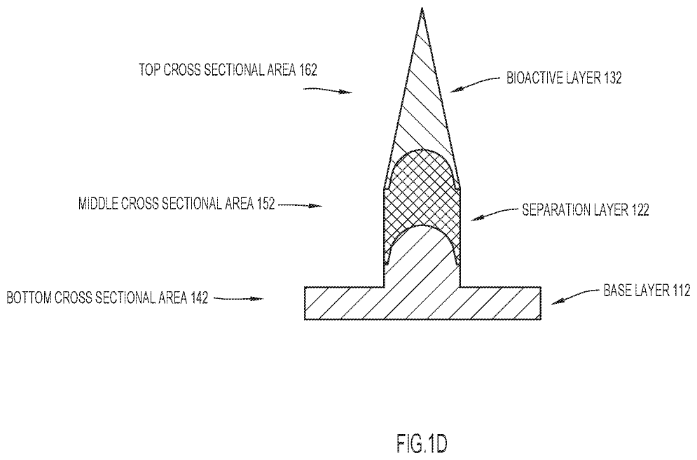

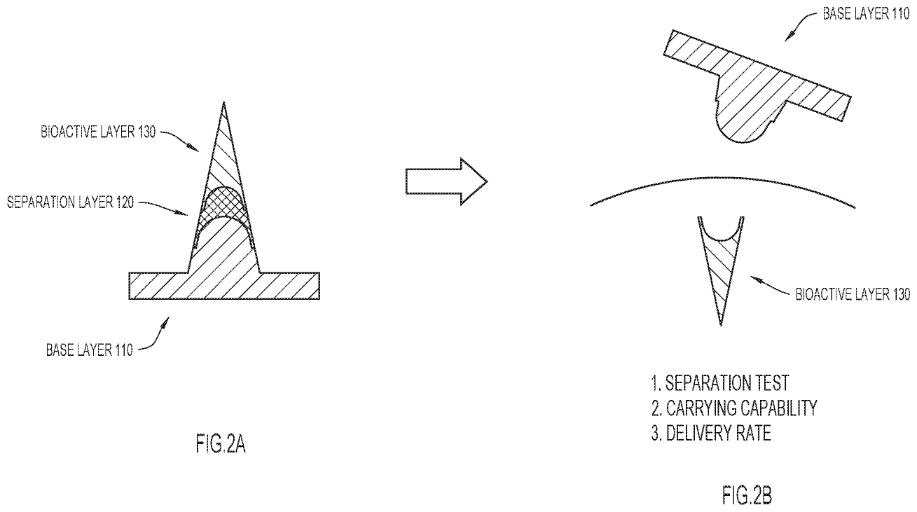

FIGS. 2A-2B show illustrations of an example mode of operation of a microneedle. FIG. 2A shows three layers of a microneedle, including base layer 110, separation layer 120, and bioactive layer 130. When exposed to physiological conditions, the separation layer 120 dissolves, leaving the bioactive layer 130 embedded in the skin, while the base layer 110 is removed.

The microneedle array can be configured to deliver a specific dose of a bioactive compound, e.g., by distributing the desired dose across each microneedle of a microneedle array. To adjust the dosage, additional microneedles may be added or removed, or alternatively, the amount of bioactive compound in each microneedle may be increased or decreased.

In some examples, the melting point of the separation layer ranges from about 30° C. to about 43° C. or from about 38° C. to about 43° C.

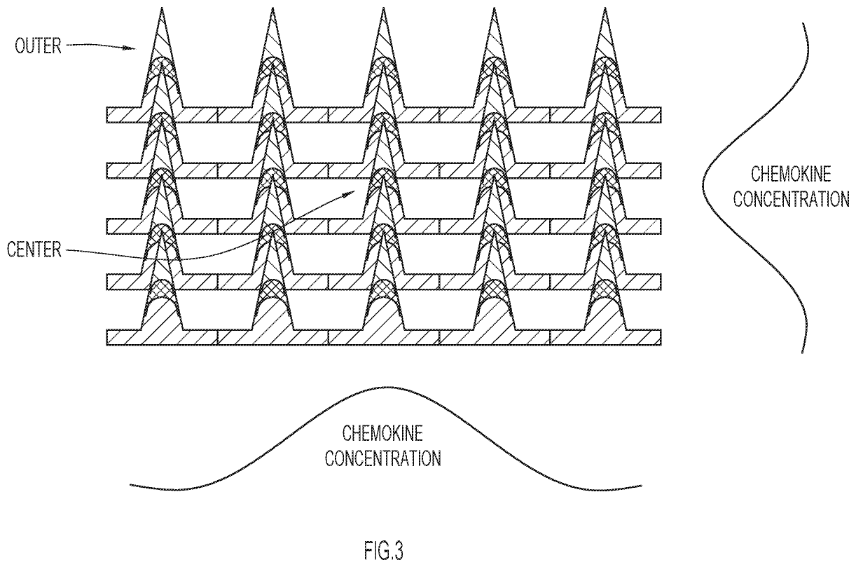

FIG. 3 shows a microneedle array with a chemokine gradient. One or more chemokines may be included in bioactive layer 130 in varying concentrations. By using lower concentrations of chemokine near the outer regions of the microneedle array and increasing the concentration of chemokine toward the center of the array, such that the highest concentration of chemokine is found at or near the center of the microneedle array, a gradient concentration can be formed, directing immune cells to the center of the microarray, thereby increasing the local concentration of immune cells.

In this example, a chemokine concentration gradient is present in both the x and y directions, such that the concentration of chemokine is highest in the center of the microarray, and lower along the outer portions of the microneedle array.

For natural killer cells (NK cells), chemokines for various receptors expressed by NK cells are shown in Table 1 (Bernardini et al., Front Immunol. 2016; 7: 402).

TABLE 1

| Chemokine | |
|---|---|
| receptor | Chemokine ligand |
| CCR1 | CCL3/MIP-1α, CCL5/RANTES, CCL7/MCP-3, |
| | CCL9/CCL10/MIP-1γ, CCL14/HCC-1, CCL15/HCC-2, |
| | CCL16/HCC-4, CCL23/MPIF-1 |
| CCR2 | CCL2/MCP-1, CCL7/MCP-3, CCL12, CCL13/MCP-4, |
| | CCL16/HCC-4 |
| CCR5 | CCL3/MIP-1α, CCL4/MIP-1β, CCL5/RANTES, |
| | CCL8/MCP-2, CCL14/HCC-1 |
| CCR7 | CCL19/MIP-3β/ELC, CCL21/SLC |
| CXCR1 | CXCL8/IL-8 |
| CXCR3 | CXCL9/Mig, CXCL10/IP-10, CXCL11/I-TAC |
| CXCR4 | CXCL12/SDF-1 |
| CXCR6 | CXCL16/SR-PSOX |
| CX3CR1 | CX3CL1/fractalkine |

Thus, any of the chemokines in Table 1 can be incorporated into the bioactive layer to direct NK cell movement towards the center of the microarray. This example is not intended to be limiting with respect to NK cells, as the chemokines in Table 1 may induce chemotaxis in other types of immune cells as well. In other embodiments, CXCL14 (a chemokine) may be selected to recruit immunocompetent cells. In some aspects, CXCL14 acts as a chemoattractant and activator of dendritic cells, may stimulate the migration of activated NK cells, and may activate monocytes in the presence of prostaglandin-E2 as well as promote chemotaxis in monocytes. In other aspects, varying concentration gradients may be established at multiple regions of the microneedle array to locally increase the concentration of immune cells at these regions.

### Compositions

In some embodiments, the bioactive component is a pharmaceutical composition, comprising a therapeutic substance or drug. Therapeutic substances, which exert actions on a living body, include DNA, RNA, peptides, proteins and corresponding derivatives, small molecules, antibodies, etc.

Antibody generally refers to immunoglobulin molecules and immunologically active portions or fragments thereof of immunoglobulin molecules, i.e., molecules that contain an antigen binding site that immunospecifically binds to an antigen (e.g., on the surface of the cell). Specifically binds typically refers to non-covalent interactions between a target entity and a binding agent and usually refers to the presence of such an interaction with a particular structural feature (e.g., such as an antigenic determinant) of the target entity with the binding agent. As understood by one of skill in the art, an interaction is considered to be specific if it occurs in the presence of other alternative interactions.

Unless the context dictates otherwise, an antibody includes but is not limited to all isotypes and subtypes of antibodies (e.g., IgA, IgD, IgE, IgG, IgM, etc.), any class (e.g., IgG1, IgG2, IgG3, IgG4, IgA1, and IgA2) or subclass of immunoglobulin molecule, as well as all active fragments (having immunological activity) thereof. It is also understood that any heavy chain (e.g., IgA, IgD, IgE, IgG, IgM) may be paired with any light chain (e.g., kappa or lambda forms).

Antibodies also include, but are not limited to, monoclonal antibodies, polyclonal antibodies, human antibodies, humanized antibodies, murine antibodies, conjugated antibodies (e.g., to a chemotherapeutic agent, to a radionuclide, to another protein, etc.), synthetic antibodies, bi-specific antibodies, chimeric antibodies, single chain antibodies, antibody fragments produced by a Fab expression library, and antibody fragments produced by mRNA display or phage display. Antibodies also include but are not limited to monovalent immunoglobulins (e.g., IgG), and fragments, e.g., F(ab′)₂, Fab₂, Fab′, Fab, Fv, single-chain Fv (scFv), scFv-Fc, VhH, disulfide-linked Fvs (sdFv), etc. or any active fragment thereof.

Examples of therapeutic drugs include, but are not limited to: α-interferon, β-interferon, erythropoietin, follitropin β, follitropin α, G-CSF, GM-CSF, human chorionic gonadotrophin, leutinizing hormone, glucagon, GNRH antagonist, human growth hormone, filgrastim, heparin, low-molecular-weight heparin, somatropin, incretin, insulin, and GLP-1 derivatives, etc.

Other examples of therapeutic drugs include but are not limited to: anti-allergic agents (e.g., azelastine hydrochloride, and ketotifen fumarate); anti-arrhythmic agents (e.g., alprenolol hydrochloride, disopyramide, mexiletine hydrochloride, nadolol, procainamide hydrochloride, and propranolol hydrochloride); anti-epileptic drugs (e.g., sodium valproate, clonazepam, and carbamazepine); anti-histamines (e.g., clemastine fumarate, chlorpheniramine maleate, diphenhydramine tannate, diphenylpyraline hydrochloride, and promethazine); anti-migraine drugs (e.g., cyproheptadine hydrochloride, dihydroergotamine mesilate, ergotamine tartrate, flunarizine hydrochloride, and sumatriptan); anti-inflammatory and analgesic drugs (e.g., acetaminophen, aspirin, indomethacin, naproxen); anti-ulcer agents (e.g., cyclophosphamide, fluorouracil, fluridine, irinotecan hydrochloride, procarbazine hydrochloride, ranimustine, and tegafur); anti-viral agents (e.g., acyclovir).

Still other examples of therapeutic drugs include but are not limited to: antibiotics (e.g., ampicillin sodium, bacampicillin hydrochloride, benzylpenicillin potassium, cephaloridine, cefdinir, cefpodoxime proxetil, cefaclor, clarithromycin, cycloserine, erythromycin, kanamycin sulfate, methyl erythromycin, propicillin potassium, and tetracycline); blood clotting promoting agents (e.g., ticlopidine hydrochloride, and warfarin potassium); cardiac stimulants (e.g., isoprenaline hydrochloride, and dopamine hydrochloride); coronary vasodilators (e.g., diltiazem hydrochloride, isosorbide dinitrate, nitroglycerine, nicorandil, and verapamil hydrochloride); chemotherapy agents (e.g., amikacin, aminosalicylic acid, capreomycin, cycloserine, ethambutol, ethionamide, isoniazid, kanamycin, pyrazinamide, rifamycins (i.e., rifampin, rifapentine, and rifabutin), streptomycin, and taxol); circulatory drugs (e.g., atenolol, captopril, flunarizine hydrochloride, nicardipine hydrochloride, losartan potassium, lisinopril, nitrendipine, trandolapril, and valsartan); diuretic agents (e.g., hydroflumethiazide, and furosemide); hormone drugs (e.g., estradiol, estriol, norethisterone acetate, metenolone acetate, progesterone, and testosterone); hypoglycemic agents (e.g., buformine hydrochloride, chlorpropamide, glibenclamide, glymidine sodium, glybuzole, and tolbutamide); peptic ulcer treating agents (e.g., cetraxate hydrochloride, cimetidine, glycopyrronium bromide proglumide, and spizofurone); peripheral vasodilators (e.g., nicametate citrate, and tolazoline hydrochloride); psychoneurotic agents (e.g., alprazolam, amitriptyline, amoxapine, diazepam, fluoxetine hydrochloride, fluvoxamine maleate, lofepramine, maprotiline, mianserin, milnacipran, paroxetine hydrochloride, setiptiline, trazodone, and venlafaxine); respiratory stimulants (e.g., dimorpholamine, lobeline hydrochloride, and naloxone hydrochloride); serotonin receptor antagonism agents (e.g., azasetron hydrochloride, granisetron hydrochloride, ondansetron hydrochloride, and ramosetron hydrochloride); skeletal muscle relaxant agents (e.g., eperisone hydrochloride, pridinol mesylate, suxamethonium hydrochloride, and tizanidine hydrochloride); sleeping/sedative drugs (e.g., amobarbital, flurazepam hydrochloride, phenobarbital, and rilmazafone hydrochloride); smoking cessation drugs (e.g., nicotine); steroid-type anti-inflammatory agents (e.g., betamethasone, dexamethasone, hydrocortisone, and prednisolone); and topical anesthetic agents (e.g., lidocaine hydrochloride, procaine hydrochloride, tetracaine hydrochloride, and propitocaine hydrochloride).

Therapeutic drugs may be used solely or in combination of two or more drugs. Pharmaceutically acceptable salts of the therapeutic drugs, or drugs in the form of inorganic and organic salts, are also contemplated herein.

The microneedle arrays described herein are suitable for use in delivering chemotherapeutics to cutaneous tumors, including skin derived tumors (e.g., basal cell, Merkel cell, melanomas, squamous cell, etc.) and metastatic tumors appearing on or near the skin. In such situations, topical delivery of a chemotherapeutic can be an effective method of treatment.

Delivery of a chemotherapeutic agent results in the apoptosis and death of skin cancer cells. The microneedle arrays of the present disclosure can be used as an alternative to or in addition to traditional topical chemotherapy approaches. Tumor antigens from apoptotic tumor cells may be presented to the immune system, inducing a local and systemic anti-tumor immune response that rejects residual tumor cells at the site of implantation of the microneedle array as well as throughout the entire body.

Multiple bioactive compounds can be delivered in a single microneedle array, enabling an immunochemotherapeutic approach based on the co-delivery of a chemotherapeutic agent with an immune stimulant (adjuvants) and/or an immune regulatory molecule (e.g., a checkpoint inhibitor).

In other embodiments, the bioactive component is a vaccine composition. The vaccine composition may comprise: (1) a whole organism, a pathogenic virus or bacteria (live, killed or attenuated); (2) a subunit of a pathogen; (3) a peptide or protein derived from a pathogen, comprising one or more antigenic epitope(s); (4) a nucleotide sequence, such as an RNA or DNA sequence capable of encoding a peptide or protein comprising the one or more antigenic epitope(s). In other embodiments, the vaccine composition may comprise: (1) one or more vectors capable of delivering a nucleotide sequence encoding a peptide or protein comprising the one or more antigenic epitope(s) to a target cell (e.g., a plasmid or a bacterial, viral or yeast vector). Viral vectors include, e.g., adenoviral vectors, adeno-associated viral (AAV) vectors, baculoviral vectors, herpes viral vectors, poxvirus vectors (MVA, NYVAC, avipox viruses and the attenuated vaccinia strain M), and retroviral vectors (including lentiviral vectors). Any suitable vector, compatible with mammalian systems, may be used according to the embodiments presented herein.

Examples of vaccines include Japanese encephalitis vaccine, rotavirus vaccine, Alzheimer disease vaccine, arteriosclerosis vaccine, a cancer vaccine, nicotine vaccine, diphtheria vaccine, tetanus vaccine, pertussis vaccine, Lyme disease vaccine, rabies vaccine, Diplococus pneumoniae vaccine, yellow fever vaccine, cholera vaccine, vaccinia vaccine, tuberculosis vaccine, rubella vaccine, measles vaccine, mumps vaccine, botulinus vaccine, herpes virus vaccine, other DNA vaccines, and hepatitis B-virus vaccine, etc.

In some embodiments, the pharmaceutical or vaccine composition comprises one or more additional ingredients e.g., to stabilize or preserve the activity of the pharmaceutical or vaccine, to modulate the dissolution or dispersal rate of the pharmaceutical or vaccine, to maintain sterility of the pharmaceutical or vaccine, etc. These ingredients include but are not limited to: antioxidants, bacteriostats, buffers, carbohydrates, chelating agents such as EDTA or glutathione, coloring, diluents, emulsifiers, excipients, flavoring and/or aromatic substances, lubricants, pH buffering agents, physiologically acceptable carriers, polypeptides (e.g., glycine), preservatives, proteins, salts for influencing osmotic pressure, solubilizers, stabilizers, surfactants, wetting agents, etc. Buffers include but are not limited to saline, neutral buffered saline, phosphate buffered saline, etc. Carbohydrates include but are not limited to dextrans, glucose, mannose, mannitol, sucrose, etc. Stabilizing excipients include but are not limited to sugars, carbohydrates and various polymers.

In still other embodiments, the bioactive component is an allergen. Allergens may include, but are not limited to, foreign proteins found in blood and vaccines as well as common allergens including: fur, dander, wool, dust mites, drugs (penicillin, sulfonamides, salicylates, etc.), foods (eggs, legumes (peanuts, soybeans), milk, seafood, sesame, soy, tree nuts, wheat), insect stings (bees, wasps, mosquitoes), metals (nick, chromium, cadmium), latex, wood, and pollen.

The subject or recipient may be mammalian, and in particular, a human. In other embodiments, the subject or recipient may be may include domestic or livestock animals, e.g., cats, cows, dogs, goats, guinea pigs, horses, pigs, rabbits, rodents, or sheep.

The subject or recipient may be a human individual, suffering from or at risk from contracting a particular disease. The subject or recipient may be a child or an adult. The subject or recipient may be a healthy individual, be at risk of contracting a disease or have been diagnosed with a disease.

### Fabrication

Many approaches are known for fabricating microneedle arrays, and all such methods are contemplated for use herein. Methods include using micromoulding to form microneedle arrays. Micromoulding relies on using an inverse mould which defines the surface of the microneedle. By filling the inverse mould with one or more layers of liquid polymer(s), and polymerizing each layer to convert the polymer(s) into a solid form, a microneedle can be formed. One or more bioactive compounds may be included in the bioactive layer of the microneedle.

Other methods for fabricating a microneedle array are known in the art and may include but are not limited to embossing, laser micromachining, milling, molding (e.g., thermoplastic injection molding, compression molding, etc.), photolithography (e.g., stereolithography, x-ray photolithography, etc.), silicon micromachining, wet or dry chemical etching, three dimensional printing, etc.

Silicon fabrication techniques using photolithography followed by wet (KOH) or dry etching (reactive ion etching with fluorine or other reactive gas) can be employed for glass materials. For example, a glass master can be formed by conventional photolithography, which serves as a master template for molding techniques to generate a plastic or PDMS-based device.

A microfluidics device may be fabricated in one or more layers that are joined together, e.g., by adhesives, dissolvable materials, etc. Alternatively, the microfluidics device may be fabricated using three-dimensional fabrication techniques including 3D printing, etc.

Many different configurations are possible, and all such configurations fall within the scope of the devices and methods disclosed herein. Exemplary embodiments of the disclosed method of fabrication and method of use may be described in a particular, sequential order. These examples are intended to be non-limiting, as the disclosure in some instances can also encompass operations that are performed in a different order or concurrently.

The microneedle layers may be structured to create different release profiles in different layers. For example, in some aspects, the separation layer may be designed to dissolve within seconds (e.g., within 5 seconds, within 10 seconds, within 30 seconds, within 60 seconds) after application. Additionally, the bioactive layer may be designed to dissolve in days (e.g., within one to two days, within one to three days, within one to five days, within one to seven days, within one to fourteen days). As the bioactive layer dissolves, nanoparticles comprising a bioactive compound embedded into the bioactive layer are released. The nanoparticles may be designed to release the bioactive compound in weeks to months (e.g., from one week to two weeks, from one week to three weeks, from one week to one month, from one week to two months or longer).

In other aspects, nanoparticles designed to release a bioactive compound over a time frame of over one week to two months may be embedded in the separation layer. For example, nanoparticles may be released into target tissue as the separation layer dissolves within seconds to a few minutes after application. The nanoparticles may be designed to release the bioactive compound in days, weeks or months (e.g., within one day, within two days, within seven days, from one week to two weeks, from one week to three weeks, from one week to one month, from one week to two months or longer).

In other embodiments, the nanoparticles may be embedded in the bioactive layer, wherein the bioactive layer is formulated to gradually erode (rather than dissolve) under physiological conditions, to supply a steady release of nanoparticles containing the bioactive compound into the implantation site of the skin. Unlike a dissolution profile, in which the bulk of the nanoparticles are released within a short timeframe, a gradual release limits the release of nanoparticles to those nanoparticles at or near the surface of the bioactive layer, with the nanoparticles in the interior of the bioactive layer largely shielded from physiological conditions. This allows a patient to receive a continuous dosage of the therapeutic for an extended period of time (e.g., days, weeks, or even months). In some aspects, the bioactive layer may erode over a time period of one week to two months or longer.

In other aspects, nanoparticles may be formed of a material that strongly absorbs near IR light, allowing induced photothermal release of the nanoparticles containing a bioactive compound by external illumination of the skin at the implantation site. Similar to the aforementioned cases, the nanoparticles may be present in the separation layer, the bioactive layer, or both the separation and bioactive layer, and may be released over a time frame of seconds to minutes to hours or days or longer.

It should be apparent to those skilled in the art that many more modifications besides those already described are possible without departing from the inventive concepts herein. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

The Examples provided herein are meant to assist one of ordinary skill in the art in making and using the disclosed devices and methods, and are not intended in any way to limit the scope of those devices and methods.

### EXAMPLES

### Example 1. Fabrication of Microneedle Array Device

Soft lithography may be used to generate the microneedle array device structures described herein. In general, an inverse mold is produced from a master mold, wherein the inverse mold represents the surface of the microneedle array device. Thus, the inverse PDMS mold acts as a receptacle for containing the various layers of the microneedle during fabrication of a microneedle array, as described in additional detail below.

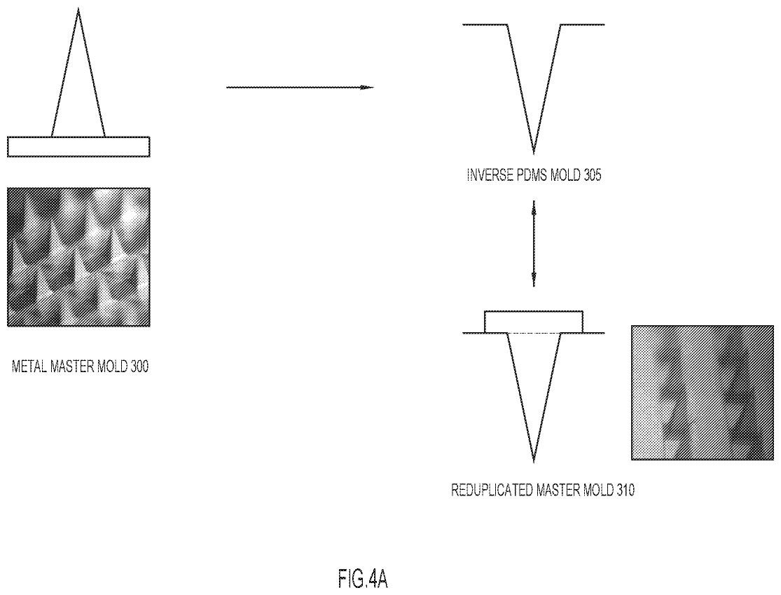

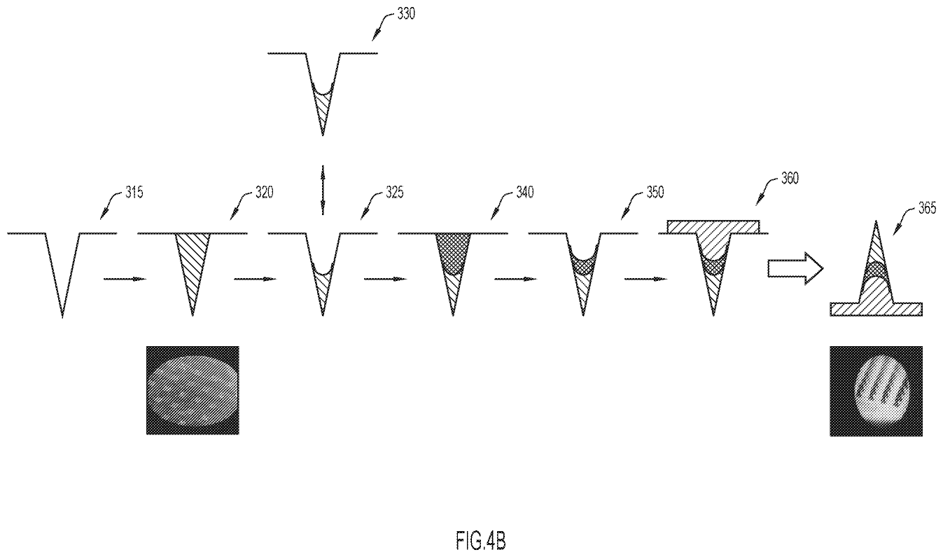

FIGS. 4A-4B show various embodiments of fabricating a microneedle device. FIG. 4A shows an example of a metal master mold 300, comprising an array of metal pyramidal extrusions from a planar plate. From this metal master mold, an inverse PDMS mold 305 may be generated, having the inverse shape of the master mold 300. To form inverse PDMS mold 305, PDMS may be poured over the metal master mold 300, or alternatively, the metal master mold may be inserted into a liquid solution of PDMS.

In one embodiment, liquid PDMS may be formed by mixing a component containing silicon hydride with another component containing a vinyl group. This mixture may be poured over the master mold or the master mold may be inserted into the liquid PDMS. The liquid PDMS is then allowed to cure or harden under conditions that are sufficient to promote solidification to form the PDMS inverse mold 305.

Once the PDMS hardens, the metal master mold 300 can be removed, leaving behind an inverse structure (the inverse PDMS mold 305) forming an array of empty pyramidal extrusions. Once formed, the inverse PDMS mold 305 may optionally be coated with silane. The inverse PDMS mold 305 acts as a receptacle for containing the various layers of the microneedle array device, during fabrication of the microneedle array device. Example fabrication steps are provided in FIG. 4B. Once separated, the master mold 300 can be reused for making additional inverse PDMS molds.

The inverse PDMS mold 305 may also be used to generate a reduplicated master mold 310, e.g., using a different material (not metal).

FIG. 4B shows an example fabrication process for generating microneedle arrays. At step 315, the inverse PDMS mold is coated with silane (e.g., various methods are known in the art for silanization of surfaces, e.g., using 5% silane in absolute ethanol for 30 minutes; 5% silane in 95% ethanol and 5% water for 10 minutes; etc.).

A PLGA solution comprising one or more bioactive components is loaded into the inverse mold at step 320, and centrifugal casting is performed to concentrate the bioactive layer into the tip of the microneedle array at step 325, and to ensure that air bubbles are eliminated from the microneedle. When filling inverse molds, a common problem is malformed or crooked microneedle tips, which typically results in microneedles that cannot penetrate the stratum corneum, e.g., due to lack of mechanical integrity or improper shape. For example, the polymers for the bioactive layer and separation layer may contain air bubbles or may not flow into the tip of the microneedle, which can lead to defects in the formation of the microneedles that undermine the structural integrity of the microneedle. Centrifugation can be used to completely fill the microneedle mold (see, Lee et al. (2008) Biomaterials 29 (13): 2113-2124).

Centrifugal casting is a process that can be used in the manufacture of micro and nano featured components to concentrate components and/or generate a bubble-free component. The centrifugation time needed to achieve removal of air bubbles is dependent upon the centrifuge's spin speed profile, the geometry of the component, and fluid properties of the solution, as well as other factors.

Once the bioactive layer is formed, an optional reflow process may be employed at step 330, which involves heating the microneedle array to about 60° C. in order to ensure that the bioactive (e.g., PLGA) layer has the desired geometry. This step map also involve a curing or hardening step in which the polymer is polymerized to form a solidified bioactive layer. Plasma treatment may be applied to the exposed surface of the PLGA layer in order to promote bonding with the separation layer. For bioactive components that are temperature sensitive, this step may be skipped.

A solution comprising PVP/PVA is loaded into the inverse mold at step 340. Centrifugal casting is performed at step 350, concentrating the separation layer, and ensuring that air bubbles are eliminated. This step map also involve a curing or hardening step in which the polymer is polymerized to form a solidified separation layer. Once the separation layer is formed, plasma treatment may be applied to the exposed surface of the separation layer, in order to promote bonding with the base layer.

A solution comprising PDMS is loaded into the inverse mold at step 360, filling the remaining portion of the microneedle (e.g., pyramidal extrusion) and forming a planar structure connecting the array of microneedles. This step map also involve a curing or hardening step in which the polymer is polymerized to form a solidified base layer. The inverse mold for the microneedle array is removed using a demolding process at step 365, and the microneedle may finish curing at room temperature.

### Example 2. Fabrication of Microneedle Array with CMC Bioactive Layer

In some embodiments, it is desirable to form a bioactive layer with CMC, e.g., a CMC-hydrogel. For example, CMC may be mixed with dH2O and with one or more bioactive components to reach about a 20-30 wt % or 25 wt % CMC concentration. The resulting mixture is stirred and equilibrated at about 4° C. for 24 hours, resulting in formation of a hydrogel. The hydrogel is degassed in a vacuum and then centrifuged at about 20,000 g to remove residual micro air bubbles.

The separation layer 120 and the base layer 110 may then be formed as previously described to construct the microneedle array.

### Example 3. In Vitro Assay for Dissolution of Separation Layer

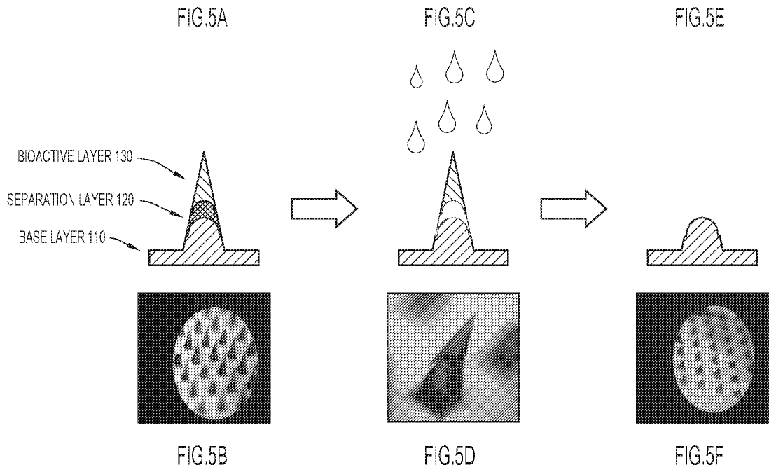

Referring to FIGS. 5A-5F, various illustrations and corresponding images are shown of a microneedle array under conditions suitable to test for dissolution of the separation layer 120 under physiological conditions or conditions that mimic one or more aspects of physiological conditions. FIG. 5A is an illustration showing an example of a single microneedle, with all three layers intact: the base layer 110 (e.g., PDMS), the separation layer 120 (e.g., PVP/PVA), and the bioactive layer 130 (PLGA). FIG. 5B is an image of a fabricated microneedle array comprising all three layers, with each individual microneedle appearing as a pyramidal protrusion from a base layer. The microneedle array was fabricated according to the techniques presented herein. FIG. 5C shows dissolution of the separation layer 120 when exposed to physiological conditions (e.g., exposure to an aqueous solution, such as distilled or deionized water, and/or a temperature of about 30-43° C. for a specified period of time, such as 5 minutes, 10 minutes, or longer). FIG. 5D is an image showing a bioactive layer that has partially detached from the base layer. FIG. 5E is an illustration showing the remaining base layer after exposure of the microneedle to physiological conditions. FIG. 5F is an image showing a microneedle array that was exposed to physiological conditions, wherein only the base layer 130 remains.

In some embodiments, plasma treatment of PLGA was shown to improve adhesion between the PLGA bioactive layer and the PVP separation layer.

### Example 4. Assay for Visualization of Microneedle Array Layers

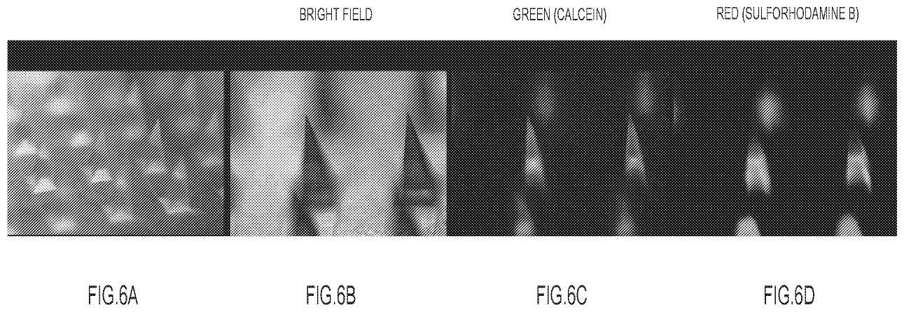

Referring to FIGS. 6A-6D, various images are shown of the microneedle array, wherein the separation layer 120 and the bioactive layer 130 were each labeled with a different fluorescent dye. FIG. 6A shows a color merged image for a plurality of microneedles with the base layer, the separation layer, and the bioactive layer. FIG. 6B shows a brightfield image of a fully assembled microneedle array comprising all three layers. FIG. 6C shows a bioactive layer 130 that was mixed with calcein (e.g., PLGA at 30 wt % mixed with calcein at 1-3 mg/mL). FIG. 6D shows separation layer 120 mixed with sulforhodamine B (e.g., PVP at 40 wt % mixed with 0.1 wt % of sulforhodamine B). These images were captured using a widefield fluorescence microscope, using filter combinations optimized for the appropriate fluorescent proteins/molecules, e.g., a red filter for sulforhodamine B and a green filter for calcein.

### Example 5. Gel Penetration and Tip Separation Assay for Microneedle Array

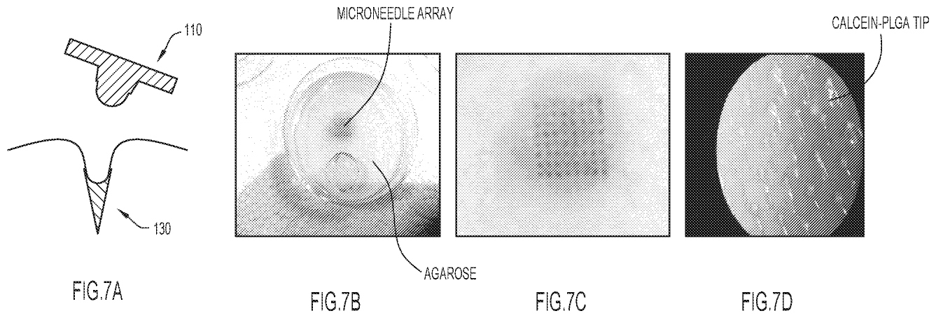

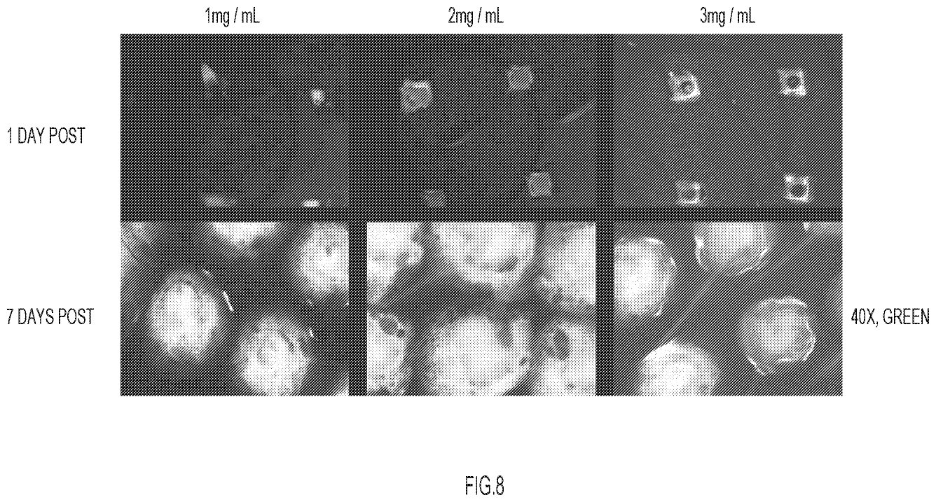

FIGS. 7A-7D show gel penetration and tip separation assays using an agarose gel platform. A 2% agarose gel was selected as a testing platform due to its mechanical similarities to human skin. In other embodiments, agarose concentrations between about 0.1% and about 10% or between about 1% and about 5% may be used for testing. In this example, a 2% (2 g/100 mL) agarose gel was used.

FIG. 7A is an illustration showing a surface (solid line), with an embedded tip (bioactive layer 130) and a separated base layer 110. FIG. 7B shows a 2% agarose gel after the microneedle array has been applied to the agarose gel. The residual dye, present in the separation layer, shows the location of the microneedle tips (corresponding to the bioactive layer). The round structure within the agarose containing dish corresponds to the base layer, after dissolution of the separation layer. FIG. 7C shows an enlarged image of the microneedle array of FIG. 7B. In this assay, the microneedle array comprising all three layers was brought into contact with a 2% agarose gel for a specified period of time (e.g., about 5 minutes or more). Pressure was applied to the microneedle array, causing the microneedle array to puncture the surface of the agarose (FIG. 7B-7C). Contact with the agarose gel, which has an aqueous component, resulted in dissolution of the separation layer 120. FIG. 7D shows embedded bioactive layers (e.g., PLGA with calcein) in the agarose gel after separation layer dissolution (e.g., PVP/PVA) and removal of the base layer. The bioactive layers (e.g., PLGA with calcein) remained embedded in the agarose gel after separation layer dissolution (e.g., PVP/PVA) and removal of the base layer (FIG. 7D).

The 2% agarose gel was created according to protocols known in the art. For example, 2.0 g of agarose (electrophoresis grade) was added to 100 ml 1×TBE electrophoresis buffer in a beaker/Erlenmeyer flask. The solution was stirred to suspend the agarose, and then heated until the agarose was dissolved. The solution was poured into Petri dishes and the agarose cooled and solidified.

### Example 6. Assay for Degradation of the Bioactive Layer

FIG. 8 shows a time series of dissolution/dispersion of the bioactive layer in a 2% agarose gel at 37° C. Images were obtained using a widefield fluorescence microscope at 40× magnification, with filter combinations optimized for the appropriate fluorescent molecules, e.g., a green filter for calcein. Microneedle arrays with varying concentrations of calcein (e.g., 1 mg/mL, 2 mg/mL, and 3 mg/mL) were formed. The microneedle array was brought into contact with the agarose gel, and pressure was applied to the microneedle array, causing the microneedle array to puncture the surface of the agarose. After a specified period of time (e.g., 5 minutes, 10 minutes, or more), the microneedle array was removed, and the agarose gel was examined to determine whether the bioactive layer was embedded in the gel, due to dispersal or dissolution of the separation layer. The agarose gel was examined one day and seven days post implantation of the bioactive layer in a 2% agarose gel. Calcein diffusion and expansion along with PLGA swelling was consistent with the degradation of PLGA as a function of time.

### Example 7. Three Dimensional Printing of Microneedle Arrays

As an alternative to molding techniques, 3D printing techniques can also be used to generate a microneedle array. This technology prints small amounts of a material (e.g., silicon, etc.), as multiple layers in an additive manner (as represented by a CAD file) to generate a three dimensional structure.

3D printing permits rapid prototyping, and can achieve a resolution between 20 and 100 μm, which is suitable for some microfluidic devices. In this example, one or more layers of the microneedle array is directly produced printed.

### Example 8. Microneedle Arrays for Allergy Treatment

A microneedle array is generated with one or more allergens in the bioactive layer. The microneedle array is brought into contact with a region of skin. Pressure is applied to the microneedle array, causing the microneedle array to puncture the surface of the skin. After a specified period of time (e.g., 5 minutes or more), the microneedle array is removed, and the remaining bioactive layer disperses at the site of insertion.

Over a period of time, the amount of allergen in the tip of each microneedle device is increased, resulting in an improvement in patient tolerance to the allergen.

### Example 9. Microneedle Arrays for Immunization

Immunization is achieved by applying antigen or vaccine containing microneedle arrays to the skin of a subject, followed by removal of the microneedle array about 5 minutes after application. Pyramidal microneedle arrays may contain 0.1 to 5 wt % of the antigen or vaccine in CMC or PLGA. After immunization, a booster may be delivered one week later, and the subjects may be assayed for immune response activity according to techniques known in the art.

In some embodiments, microneedle arrays can be configured to deliver multiple antigens to a patient, e.g., each row of microneedles or each microneedle itself may contain a different antigen or vaccine, or alternatively, an individual microneedle may contain a mixture of antigens or vaccines.

### Example 10. Microneedle Arrays for Skin Cancer Treatment

A microneedle array is generated with one or more chemotherapeutic agents in the bioactive layer. The microneedle array is brought into contact with a region of skin that contains skin cancer. Pressure is applied to the microneedle array, causing the microneedle array to puncture the surface of the skin. After a specified period of time (e.g., 5 minutes or more), the microneedle array is removed, and the remaining bioactive layer slowly disperses at the site of insertion, thereby destroying cancer cells.

In some embodiments, the chemotherapeutic is integrated at a concentration of 5 mg/g of CMC or PLGA. This amount corresponds to about 140 μg per array, which is large enough to be therapeutically relevant, but small enough to be below levels associated with systemic toxicities.

In other aspects, human skin cell cultures can be used to assess the cytotoxicty of a chemotherapeutic. The chemotherapeutic can be delivered by microneedle array according to the protocols described herein. After 72 hours of exposure to the bioactive component, the cultured skin cells can be cryo-sectioned and fixed, and apoptosis evaluated using techniques known in the art (e.g., In Situ Cell Death Detection Kit, TMR Green, Roche). Fluorescent microscopic image analysis of these sections can be used to determine the extent of apoptosis of epidermal cells.

### Example 10. Three Dimensional Printing

In another aspect, three-dimensional printing may be used to manufacture the microarray devices provided herein. Methods of printing biological substances and synthetic polymers as well as hydrogels are known in the art (see, e.g., US Patent Application No. 20170218228A1, International Application WO 2018058135A1, and US Patent Application No. 20160198576A1).

In general, the polymeric material from which the microneedle is formed may be mixed with a solvent or dispersing medium to form a liquid composition, referred to as a biosolution. The biosolution may be mixed with other biological compositions (e.g., a bioactive compound) and printed to form a three dimensional structure. In some aspects, three biosolutions may be formulated, e.g., a first biosolution that when printed forms the base layer, a second biosolution that when printed forms the separation layer, and a third biosolution layer that when printed forms the bioactive layer. These solutions may be additively printed to form the three layers of the microneedle array as provided herein. In some aspects, the base layer is printed first, the separation layer is printed on top of the base layer, and the bioactive layer is printed on top of the separation layer. In other aspects, the bioactive layer is printed first, the separation layer is printed on top of the bioactive layer, and the base layer is printed on top of the separation layer.

In general, the biosolution will have a viscosity low enough under printing conditions to pass through the nozzle of the printer head, and will solidify to a stable shape during and/or after printing, allowing layers to be additively manufactured. To form a three dimensional structure, material may be deposited onto a printable surface in an iterative manner. For example, a first sheet of the biosolution may be deposited, and once the first sheet has solidified, a second sheet may be added on top of the first sheet. This process may be repeated to build three dimensional structures (e.g., a base layer, a separation layer, a bioactive layer). Shapes may be determined by varying the dimensions of the individual printed sheets.

Typical viscosities of the biosolution (e.g., a gel or liquid form, etc.) may be in the range of about 0.5 to about 50 centipoise, from about 1 to about 20 centipoise, or from about 1 to about 10 centipoise at or about a temperature of about 18-26° C. However, viscosities of the biosolution may range from 0.5 to 1000 centipoise or more, in other aspects.

1. A microneedle array device for transdermal injection comprising a plurality of microneedles, wherein each microneedle comprises:
a base layer;
a bioactive layer comprising one or more bioactive compounds;
a separation layer that dissolves or disperses under physiological conditions, wherein the separation layer is positioned between the base layer and the bioactive layer.

2. The device of claim 1, wherein the bioactive compounds comprise at least two different bioactive compounds.

3. The device of claim 1, wherein the bioactive compounds comprise one or more compounds selected from the group consisting of: an allergen, an antibody or fragment thereof, an affirmer, an analgesic agent, an anesthetic agent, an anti-asthmatic agent, an antibiotic, an anti-depressant agent, an anti-diabetic agent, an anti-fungal agent, an antigen with an adjuvant, an antigen without an adjuvant, an anti-hypertensive agent, an anti-inflammatory agent, an anti-neoplastic agent, an aptamer, a type of bacterium, a chemotherapeutic agent, a cosmetic, DNA, a glycoprotein, an immunostimulating agent, an immunosuppressive agent, a lipid, a nucleic acid construct, a nucleotide, an oligonucleotide, an oligosaccharide, a peptide, a polysaccharide, a protein, a protein scaffold, RNA, a small molecule, a vaccine, a vaccine with an adjuvant, a vector, a viral vector, a live virus, an inactivated virus, a wound healing drug or agent, a birth control drug, and a nanoparticle.

4. The device of claim 3, wherein the cosmetic is selected from the group consisting of an anti-aging compound, an anti-wrinkle compound, a dermal filler, an ink for a tattoo, and a skin brightening compound.

5. The device of claim 1, wherein the bioactive layer additionally comprises one or more compounds selected from the group consisting of an antioxidant, a bacteriostat, a buffer, a carbohydrate, a chelating agent, a coloring, a diluent, an emulsifier, an excipient, a flavoring and/or an aromatic substance, a lubricant, a pH buffering agent, a carrier, a polypeptide, a preservative, a salt, a solubilizer, a stabilizer, a surfactant, and a wetting agent.

6. The device of claim 1, wherein the bioactive layer additionally comprises poly lactic-co-glycolic acid (PLGA).

7. The device of claim 1, wherein the bioactive layer additionally comprises carboxymethylcellulose (CMC).

8. The device of claim 1, wherein the separation layer dissolves when exposed to physiological conditions.

9. The device of claim 8, wherein the physiological conditions comprise one or more of:
(a) an aqueous environment; or
(b) a temperature of about 30° C. to about 43° C.

10. The device of claim 1, wherein the separation layer comprises polyvinyl alcohol (PVA) and/or polyvinyl pyrrolidone (PVP).

11. The device of claim 1, wherein the base layer comprises polydimethylsiloxane (PDMS).

12. The device of claim 1, wherein all or substantially all of the one or more bioactive compounds are in the bioactive layer.

13. The device of claim 1, wherein the bioactive layer has a top cross sectional area, the separation layer has a middle cross sectional area, and the base layer has a bottom cross sectional area, wherein the top cross sectional area is less than the middle cross sectional area, and the middle cross sectional area is less than the bottom cross sectional area.

14. The device of claim 1, wherein the bioactive layer has a shape that tapers to a point.

15. The device of claim 1, wherein the microneedle has a pyramidal shape or a conical shape.

16. The device of claim 1, wherein the microneedle has a height of between about 50 μm to about 2500 μm.

17. The device of claim 1, wherein the microneedle has a length or a width of between about 100 μm to about 1000 μm.

18. The device of claim 1, wherein a total length and a total width of the device ranges from about 1 to 100 mm by about 1 to 100 mm.

19. The device of claim 1, wherein the device includes at least 50 microneedles.

20. A method of fabricating a microneedle array device comprising:
(a) applying a first solution comprising one or more bioactive compounds to an inverse mold and subjecting the first solution to centrifugal casting;
(b) applying conditions suitable to cause solidification of the first solution to form a bioactive layer;
(c) applying a second solution comprising a polymer that dissolves under physiological conditions to the bioactive layer in the inverse mold and subjecting the second solution to centrifugal casting;
(d) applying conditions suitable to cause solidification of the second solution to form a separation layer;
(e) applying a third solution comprising a polymer that forms a base layer to the separation layer in the inverse mold and subjecting the third solution to centrifugal casting;
(f) applying conditions suitable to cause solidification of the third solution to form the base layer; and
(g) separating the base layer, the separation layer and the bioactive layer from the inverse mold using a demolding step to form the microneedle array device.

21. The method of claim 20, wherein the inverse mold is coated with silane.

22. The method of claim 20, wherein plasma treatment is applied to the bioactive layer to improve adhesion between the separation layer and the bioactive layer.

23. The method of claim 20, wherein plasma treatment is applied to the separation layer to improve adhesion between the separation layer and the bioactive layer.

24. The method of claim 20, further comprising exposing the bioactive layer to a temperature of about 60° C.

25. A method of inducing a biological activity in a patient using a microneedle array device, comprising:
(1) applying the microneedle array device to the skin of a patient, wherein microneedles puncture the skin;
(2) maintaining contact of the microarray device with the skin for a period of time sufficient for a separation layer of the microneedles to dissolve or disperse; and
(3) removing the microarray array device from the patient, wherein a bioactive layer remains implanted within the skin.

26. The method of claim 25, wherein the period of time is about 5 minutes or about 10 minutes.

27. The method of claim 25, wherein the microneedle array device punctures the stratum corneum layer of the skin.

28. The method of claim 25, wherein the bioactive layer comprises an allergen in a concentration sufficient to induce tolerance in the patient as part of an allergy treatment program.

29. The method of claim 25, wherein the bioactive layer comprises a vaccine in a concentration sufficient to trigger an immune response in the patient providing immunity to a pathogenic disease.

30. The method of claim 25, wherein the bioactive layer comprises a chemotherapeutic agent in an effective amount to treat skin cancer.

31. The method of claim 30, wherein the bioactive layer additionally comprises a checkpoint inhibitor.

32. The method of claim 25, wherein applying the microneedle array device to the skin of a patient results in the separation layer dissolving in less than sixty seconds, and
wherein the implanted bioactive layer comprises nanoparticles comprising a bioactive compound, such that the bioactive layer dissolves in one to seven days to release the nanoparticles and the nanoparticles dissolve in one to four weeks to release the bioactive compound.

33. The method of claim 25, wherein the separation layer additionally comprises nanoparticles comprising a bioactive compound,
wherein the nanoparticles are released into the skin when the separation layer dissolves, and
wherein the nanoparticles dissolve in one to four weeks to release the bioactive compound.

34. The method of claim 25, wherein the implanted bioactive layer comprises nanoparticles comprising a bioactive compound, wherein the bioactive layer is formulated to release nanoparticles through erosion of the surface of the bioactive layer, and
wherein the nanoparticles dissolve in one to four weeks to release the bioactive compound.

35. The method of claim 25, wherein the separation layer and/or the bioactive layer comprise(s) nanoparticles comprising a bioactive compound, such that the nanoparticles absorb near infrared (IR) light allowing photothermal release of the bioactive compound by external illumination of the skin at the site of implantation of the microneedle.

36. The method of claim 25, wherein the microneedle array device additionally comprises a chemokine within the bioactive layer, configured to form a gradient concentration of the chemokine upon implantation of the bioactive layer within the skin.

37. The method of claim 36, wherein the chemokine induces the chemotaxis of natural killer (NK) cells towards the implanted bioactive layer.

38. A method of manufacturing a microneedle array device using three dimensional printing techniques comprising:
printing a first biosolution comprising a polymer onto a surface to form a base layer;
printing a second biosolution comprising a polymer that dissolves under physiological conditions onto the solidified base layer to form a separation layer;
printing a third biosolution comprising a polymer and one or more bioactive compounds onto the solidified separation layer to form a bioactive layer,
wherein the base layer, separation layer, and bioactive layer form the shape of a pyramid, and wherein the base layer, separation layer, and bioactive layer may each be printed by deposition of one or more sheets of the respective biosolution.
